(19) United States
(12) Patent Application Publication
(10) Pub. No.: US 2019/0076081 A1
(43) Pub. Date: Mar. 14, 2019

(54) SYSTEMS, COMPOSITIONS, AND METHODS FOR TARGETED CHALLENGE AND IDENTIFICATION OF GUT MICROBIOTA

(71) Applicant: ELWHA LLC, Bellevue, WA (US)

(72) Inventors: Roderick A. Hyde, Redmond, WA (US); Gary L. McKnight, Bothell, WA (US); Elizabeth A. Sweeney, Seattle, WA (US)

(21) Appl. No.: 15/701,988

(22) Filed: Sep. 12, 2017

(51) Int. Cl. A61B 5/00; A61B 5/1455; A61B 5/145; A61B 5/1495

(52) U.S. Cl. CPC A61B 5/4283; A61B 5/4238; A61B 5/4255; A61B 2010/0061; A61B 5/14507; A61B 5/14539; A61B 5/1495; A61B 5/14552

(57) ABSTRACT

Identifying gut microbiota in a specific site of the gut through production of a measurable response following introduction of a challenge agent to the specific site of the gut is disclosed. A composition embodiment includes, but is not limited to, a challenge agent configured to elicit a measurable response by the at least one targeted microbe at the specific site in the gut, the measurable response detectable in a bodily gaseous emission; and a controlled release system encompassing at least a portion of the challenge agent, the controlled release system configured to access the specific site of the gut.

If an Application Data Sheet (ADS) has been filed on the filing date of this application, it is incorporated by reference herein. Any applications claimed on the ADS for priority under 35 U.S.C. §§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC § 119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In an aspect, a composition for detecting at least one targeted microbe in a specific site of the gut includes, but is not limited to, a challenge agent configured to elicit a measurable response by the at least one targeted microbe at the specific site in the gut, the measurable response detectable in a bodily gaseous emission; and a controlled release system encompassing at least a portion of the challenge agent, the controlled release system configured to access the specific site of the gut.

In an aspect, a method for detecting at least one targeted microbe in a specific site of the gut includes, but is not limited to, targeting at least one microbe of the gut with a challenge composition configured for delivery to the specific site of the gut, the challenge composition configured to elicit a measurable response by the at least one targeted microbe detectable in a biofluid; and detecting the measurable response from the biofluid.

In an aspect, a composition for detecting at least one targeted microbe in a specific site of the gut includes, but is not limited to, a challenge agent configured to elicit a measurable response by the at least one targeted microbe at the specific location in the gut, the measurable response detectable in a biofluid; and a controlled release system encompassing at least a portion of the challenge agent, the controlled release system configured to access the specific location of the gut.

In an aspect, a composition for detecting at least one targeted pathogen in a specific site of the gut includes, but is not limited to, a challenge agent configured to elicit a measurable response by the at least one targeted pathogen at the specific site in the gut, the measurable response detectable in a biofluid; and a controlled release system encompassing at least a portion of the challenge agent, the controlled release system configured to access the specific location of the gut.

In an aspect, a system includes, but is not limited to, a composition for detecting at least one targeted microbe in a specific site of the gut, the composition including a challenge agent configured to elicit a measurable response by the at least one targeted microbe at the specific site in the gut, the measurable response detectable in a bodily gaseous emission; and a controlled release system encompassing at least a portion of the challenge agent, the controlled release system configured to access the specific site of the gut. The system also includes, but is not limited to, an emissions analysis device including a body structure defining an orifice configured to receive the bodily gaseous emission; a sensor operably coupled to the orifice and configured to detect at least one analyte from the bodily gaseous emission and generate one or more sense signals, the one or more sense signals associated with the measurable response; circuitry operably coupled to the sensor and configured to receive the one or more sense signals associated with the measurable response; and a reporter operably coupled to the circuitry and configured to generate one or more communication signals associated with the at least one targeted microbe.

In an aspect, a method for detecting at least one targeted microbe in a specific site of the gut includes, but is not limited to, targeting at least one targeted microbe of the gut with a challenge composition configured for delivery to the specific site of the gut, the challenge composition configured to elicit a measurable response by the at least one targeted microbe; receiving, via an emissions analysis device, a biofluid containing at least a portion of the measurable response; detecting, via the emissions analysis device, at least one analyte from the biofluid; generating one or more sense signals associated with the measurable response subsequent to detecting the at least one analyte from the biofluid; and generating one or more communication signals associated with the at least one targeted microbe.

In an aspect, a system includes, but is not limited to, a composition for detecting at least one targeted microbe in a specific site of the gut, the composition including a challenge agent configured to elicit a measurable response by the at least one targeted microbe at the specific site in the gut, the measurable response detectable in a biofluid; and a controlled release system encompassing at least a portion of the challenge agent, the controlled release system configured to access the specific site of the gut. The system also includes, but is not limited to, an emissions analysis device including a body structure defining an orifice configured to receive the biofluid; a sensor operably coupled to the orifice and configured to detect at least one analyte from the biofluid and generate one or more sense signals, the one or more sense signals associated with the measurable response; circuitry operably coupled to the sensor and configured to receive the one or more sense signals associated with the measurable response; and a reporter operably coupled to the circuitry and configured to generate one or more communication signals associated with the at least one targeted microbe.

In an aspect, a system includes, but is not limited to, a composition for detecting at least one targeted pathogen in a specific site of the gut, the composition including a challenge agent configured to elicit a measurable response by the at least one targeted pathogen at the specific site in the gut, the measurable response detectable in a biofluid; and a controlled release system encompassing at least a portion of the challenge agent, the controlled release system configured to access the specific site of the gut. The system also includes, but is not limited to, an emissions analysis device including a body structure defining an orifice configured to receive the biofluid; a sensor operably coupled to the orifice and configured to detect at least one analyte from the biofluid and generate one or more sense signals, the one or more sense signals associated with the measurable response; circuitry operably coupled to the sensor and configured to receive the one or more sense signals associated with the measurable response; and a reporter operably coupled to the circuitry and configured to generate one or more communication signals associated with the at least one targeted pathogen.

In an aspect, a system includes, but is not limited to, a composition for detecting at least one targeted microbe in a specific site of the gut, the composition including a challenge agent configured to elicit a measurable response by the at least one targeted microbe at the specific site in the gut, the measurable response detectable in an oral gaseous emission; and a controlled release system encompassing at least a portion of the challenge agent, the controlled release system configured to access the specific site of the gut. The system also includes, but is not limited to, a breathalyzer device including a body structure defining an orifice configured to receive the oral gaseous emission; a sensor operably coupled to the orifice and configured to detect at least one analyte from the oral gaseous emission and generate one or more sense signals, the one or more sense signals associated with the measurable response; circuitry operably coupled to the sensor and configured to receive the one or more sense signals associated with the measurable response; and a reporter operably coupled to the circuitry and configured to generate one or more communication signals associated with the at least one targeted microbe.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic of a composition for detecting at least one targeted pathogen in a specific site of the gut.

FIG. 2 is a schematic of an embodiment of a composition such as shown in FIG.

FIG. 3A is a schematic of an embodiment of a composition such as shown in FIG. 1.

FIG. 3B is a schematic of an embodiment of a composition such as shown in FIG. 1.

FIG. 4 is a schematic of a system for detecting at least one targeted pathogen in a specific site of the gut via production of a measurable response by the at least one targeted pathogen following introduction of a challenge agent to the specific site of the gut.

FIG. 5 is a schematic of an embodiment of a system such as shown in FIG. 4.

FIG. 6 is a schematic of an embodiment of a system such as shown in FIG. 4.

FIG. 7 is a schematic of an embodiment of a system such as shown in FIG. 4.

FIG. 8 is a schematic of an embodiment of a system such as shown in FIG. 4.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Diagnostic tests utilizing analysis of a biofluid are considered highly desirable as being inexpensive and noninvasive. However, the nonlocalized nature of biofluids leads to difficulties that limit employment and interpretation of such tests. In particular, tests utilizing challenge compounds face limitations of aligning challenge and tested variable. A challenge substance can be processed by a microbe (e.g., a colonic anaerobe), producing analytes measurable in biofluids, for example directly in bodily gas (e.g., breath or flatus), after being absorbed into the bloodstream and released in exhalant via the lungs, or in bodily excretions. The exemplary breath testing in gut disorders measures analytes in exhalant, produced by gut microbes exposed to a challenge substance. Results can be interpreted as indicative of disorders as disparate as microbial dysbiosis and nutrient maladsorption. If the microbe is present in an earlier part of the gut, the analyte appears in exhalant earlier, if the microbe is present farther down the gut and the challenge is still available for processing (i.e., is not absorbed), then the analyte appears later, indicating maladsorption. Such indirect and imprecise results limit both the specificity and the sensitivity of this type of analysis.

Systems, compositions, and methods are described for detecting and identifying gut microbiota through introduction of a challenge composition to a specific site of the gut. The challenge composition can elicit a measurable response by at least one pathogen. The challenge composition can elicit a measurable response by at least one microbe, for example a pathogenic microbe or a commensal microbe, at the specific site of the gut, where such measurable response is detectable in a biofluid (e.g., bodily gaseous emission, blood, bodily excretion, etc.), such as via an emissions analysis device. The measurable response can include distinctive compositions of analytes to identify the source of the measurable response as being a specific microbe, belonging to a particular microbe genus, or the like. The composition of the gut microbiota, including the presence and location of particular targeted microbes, can provide information for the diagnosis of health conditions, diseases, disease progression, risk of disease, and the like. Moreover, compositions or changes in composition (quantitative or qualitative), or temporal aspects of measurable responses to a challenge can serve as biomarkers for identifying particular microbes present in the gut (e.g., present at a specific site in the gut), for identifying certain diseases, and the like. For example, concentrations of hydrogen sulfide can be correlated with pathogenic microbes including bacteria associated with halitosis; concentrations of ammonia and carbon dioxide following challenge with urea can be correlated with Helicobacter pylori associated with a stomach ulcer; concentrations of ethanol following a challenge with glucose can be correlated with overgrowth of Candida species; concentrations of ammonia after challenge with urea can be correlated with a fungal infection (e.g., by Cryptococcus neoformans), for example in the esophagus; or concentrations of hydrogen following lactose challenge can be temporally correlated with abnormal growth of anaerobic bacteria in the small intestine. Identification of the gut microbiota through a measurable response to a challenge, detectable in a biofluid such as a bodily gaseous emission, can provide a noninvasive and convenient way to determine a disease state in an individual, determine a progression of a disease over time, and the like.

The systems, compositions, and methods described herein can include a composition to detect at least one targeted microbe in a specific site of the gut by incorporating a challenge agent and a controlled release system. The challenge agent is configured to elicit a measurable response by the at least one targeted microbe at the specific site of the gut, where such measurable response is detectable in a biofluid (e.g., bodily gaseous emission, blood, bodily excretion, etc.). For example, the specific site of the gut can include, but is not limited to, the mouth, throat, esophagus, stomach, duodenum, jejunum, proximal ileum, distal ileum, colon, rectum or any portion of the alimentary canal. In an example, the specific site of the gut can include one or more biomarkers (e.g., a gut biomarker, cell surface marker, pH marker, chemical marker, extracellular matrix component, cellular secretion, etc.). The detection can be facilitated by an emissions analysis device, such as an endoluminal device, an intraoral device, an ex vivo device, a chromatograph, a chip sensor, an electrochemical sensor, a chemo-resistive gas sensor, an optic sensor, etc. The challenge agent can be delivered to the specific site by an at least partially intraluminal device, an endoluminal device, an intraluminal endoscope, a capsule pillcam/endoscope, a lumen -traveling device, or the like. The challenge agent can include, but is not limited to, at least one saccharide (e.g., fructose, lactose, glucose, sucrose, etc.), at least one taggant (e.g., a radioisotope, a fluorescent tag, a mass tag, a chemical tag (e.g., a volatile organic compound), etc.), at least one substrate configured to interact with the at least one targeted microbe in a cell process (e.g., a metabolic substrate, an enzymatic substrate, or a substrate including a taggant, such as a releasable taggant).

The controlled release system of the composition encompasses at least a portion of the challenge agent, and facilitates transfer of the challenge agent to the specific site of the gut. For example, if the specific site of the gut is the stomach, the controlled release system can transport the challenge agent through the mouth, throat, and esophagus to deposit the challenge agent to the stomach to elicit a measurable response from at least one target microbe in the stomach. The controlled release system can include, but is not limited to, a capsule, an encapsulation structure, a controlled release coating, a controlled release matrix, a polymer, a gelling agent, a first controlled release component and at least one additional controlled release component, at least one pH-sensitive component (e.g., acid-resistant component, acid-sensitive component, etc.), an extended-release component, a delayed-release component, a target-release component, a pH-modulated system, an enteric system, a time controlled system, a microbially controlled system, a luminal pressure-controlled system, at least one responsively controlled system, a synthetic component, an electroactive component, an electrochemical component, a magnetic component, a polysaccharide component, a lipid component (e.g., lipid micelle, liposome, etc.), a protein component, a hydrophobic component, a hydrophilic component, a component digestible by one or more resident microbes at the specific site of the gut (e.g., a polysaccharide component (e.g., trehalose), a lipid component, or a protein component), a recognition element to detect a biomarker present in the specific site of the gut (e.g., a binding agent, a ligand, an antibody, etc.), a substrate specific for a resident microbial enzyme, an enzyme-degradable casing, or the like. The controlled release system can be configured to remain at a target gut site for a period that extends beyond a normal retention time for material within the individual's gut. For instance, the controlled release system can include a gastroretentive component, an intestine-retentive component, a density that is less than a density of one or more digestive fluids in the target gut site, a density that is more than a density of one or more digestive fluids in the target gut site, a bioadhesive component, a swellable component, an extendable component, an unfoldable component, a floating component, an adherent component, or the like. The composition can include, but is not limited to, at least one of one or more liquid, one or more solid, one or more suspension, one or more gel, or one or more emulsion.

The composition can be introduced to an individual subject according to various introduction methods based on the specific site of the gut. For example, the composition can be introduced via an oral route, a rectal route, an injection, an implant, an endoscope (e.g., an intraluminal endoscope or a capsule endoscope), or the like. Upon reaching the specific site of the gut, the challenge agent elicits the measurable response by the at least one targeted microbe for detection in a biofluid of the individual. The system and methods described herein can include an emissions detection device to receive at least a portion of the biofluid and detect at least one analyte from the biofluid. The emissions detection device (or remote or external device, or combinations thereof) can process the detected analytes to identify one or more microbes present at the specific site. For example, the emissions analysis device can employ a body structure, a sensor, circuitry, and a reporter to receive the biofluid; sense at least one analyte in the biofluid; process information associated with the analyte; provide communication regarding the measurable response, the targeted microbe; etc.; or combinations thereof. The emissions analysis device can be configured as an endoluminal device, an intraoral device, an ex vivo device, a breathalyzer device, a portable device, a hand-held device, or combinations thereof. The sensor (or sensor assembly) of the emissions analysis device can include, but is not limited to, a chromatograph, a chip sensor, an electrochemical sensor, a chemo-resistive gas sensor, an optic sensor, a nuclear magnetic resonance (NMR) instrument, or combinations thereof. The emissions analysis device can include at least one optical component to identify a biomarker associated with the specific site of the gut. For example, the emissions analysis device can be at least one of an endoluminal device or an intraoral device configured to identify the specific site of the gut, where the controlled release system can access the specific site of the gut identified by the emissions analysis device (e.g., delivered on-site).

The emissions analysis device can be operably coupled with an external device, such as for facilitating processing of information, transfer of communications, display of information, or the like. For example, the reporter of the emissions analysis device can be operably coupled to the external device, such as via one or more transmitters, transceivers, etc. The external device can include, but is not limited to, a mobile communication device, a mobile platform, a mobile healthcare platform, a computing device (e.g., PC, a tablet, or a cell phone), a kiosk, a device supporting an external network (e.g., an external health network), or the like. At least one of the emissions analysis device or the external device can include a recommendation module for providing analysis and recommendations associated with information sensed by the sensor of the emissions analysis device. Additionally or alternatively, the external device can provide a product for consumption by the individual to aid in altering the microbial distribution within the gut responsive to communications from the recommendation module, the circuitry can communicate with a source of products to order a recommended product (e.g., for purchase), or the like. The reporter can provide information to the individual subject and/or another party, such as by including one or more of a display device (e.g., to provide a visual indication pertaining to the targeted microbe, analytes detected, etc.), an auditory device (e.g., to provide an auditory indication pertaining to the targeted microbe, analytes detected, etc.), a vibration device (e.g., to provide a vibratory indication pertaining to the targeted microbe, analytes detected, etc.), a transmitter or transceiver (e.g., to provide one or more communication signals pertaining to the targeted microbe, analytes detected, etc.). The emissions analysis device can include a user interface to provide interactivity between the system and the individual subject or other party (e.g., health care provider, insurance company representative, etc.). For example, the user interface can receive a user input from an individual subject, display an output of the one or more communication signals associated with the at least one targeted microbe, display an output associated with an operation of the emissions analysis device, or combinations thereof. The output from the user interface can include, but is not limited to, visual output (e.g., text, graphics, etc.), audio output, tactile output, or combinations thereof.

The circuitry of the emissions analysis device can facilitate processing of data obtained by the sensors. For example, the circuitry can include a comparison module to compare sense signals generated by the sensor to reference data (e.g., stored in memory, accessible from the external device, uploaded via the user interface, etc.). The reference data can include, but is not limited to, microbe types, microbiome distributions, correlations between microbe type and detectable analyte, correlations between microbiome distribution and detectable analyte, correlations between microbe type and a risk of disease or physiological disorder (e.g., infection or microbial overgrowth, inflammation, autoimmunity, metabolic disorders (e.g., obesity), cardiac disorders, circulatory disorders, mental disorders, emotional disorders, etc.), correlations between microbiome distribution and a risk of disease or physiological disorder (e.g., infection or microbial overgrowth, inflammation, autoimmunity, metabolic disorders (e.g., obesity), cardiac disorders, circulatory disorders, mental disorders, emotional disorders, etc.), correlations between microbe type and a physiological benefit (e.g., treatment or prevention of pathogenic colonization, metabolic benefits, etc.), correlations between microbiome distribution and a physiological benefit (e.g., treatment or prevention of pathogenic colonization, metabolic benefits, etc.), correlations between microbe type and drug uptake or efficacy, correlations between microbiome distribution and drug uptake or efficacy, correlations between microbe type and population data, correlations between microbiome distribution and population data, or the like. In embodiments, the reference data can include, but is not limited to, a presence of a disease or physiological disorder, staging of a disease or physiological disorder, or combinations thereof. The circuitry can also include a recommendation module to provide recommendations and other information to the individual subject, to a third party (e.g., health care provider, insurance company representative, researcher), etc. after comparison by the circuitry of the sense signals from the sensor to the reference data. For example, the comparison module can compare the sense signals from the sensor to the reference data over time (e.g., at a plurality of differing points of time), and the recommendation module can provide a recommendation to alter a microbial population of the specific site of the gut. Such a recommendation can include a recommendation to increase a particular microbe population, a recommendation to decrease a particular microbe population, a recommendation to communicate with a healthcare professional or public health official (or directly establishing a communication to the healthcare professional or public health official), a recommendation to change a diet, a recommendation to introduce a food or drink, a recommendation to introduce a probiotic, a recommendation to introduce a prebiotic, a recommendation to introduce a specific microbe (e.g., a commercially available microbe, a stored microbe (e.g., pre-operative sample), a microbe collected from a specific individual (e.g., a family member or a celebrity) or type of individual (e.g., having a specific trait such as high metabolism), a genetically engineered microbe, or the like), a recommendation for a fecal transplant (e.g., from a user's frozen sample, a family member, etc., such as by introduction of a washed sample via endoscope), a recommendation to introduce a nutrient or nutraceutical, a recommendation to undergo a therapeutic treatment (e.g., antimicrobial treatment, anti-inflammatory treatment, etc.), a recommendation to alter a therapeutic treatment, a recommendation to increase physical activity, or the like, or combinations thereof. For example, the comparison module can compare the sense signals from the sensor to the reference data that includes microbe or microbiome data from multiple users or a user population at a single time point or over time, and make a recommendation. Such a recommendation, for instance, can include information regarding the health of a population (e.g., outbreak of a pathogen) or the response of a population (e.g., response to a shared food such as to identify a pathogen or allergen, or response to a shared situation).

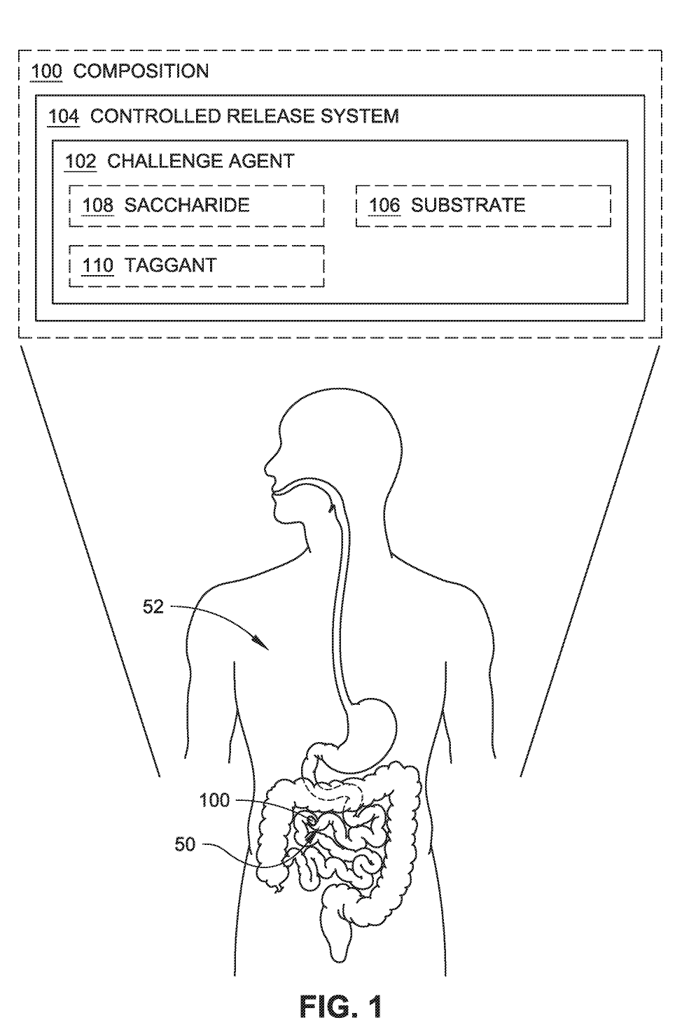

In embodiments, shown in FIG. 1, a composition 100 is configured to facilitate detection of at least one microbe, such as a commensal microbe, or at least one targeted pathogen, such as a pathogenic microbe, in a specific site 50 of a gut 52 of an individual subject. While the specific site 50 is shown in FIG. 1 as a portion of the small intestine, it is noted that the specific site 50 can be any portion or portions of the gut 52, and not limited to the example specific site 50 shown. In embodiments, the composition 100 can be delivered to the individual subject via at least one of an oral route, a rectal route, an injection, an implant, or an endoscope. For example, specific site 50 of the gut can include, but is not limited to, the mouth, throat, esophagus, stomach, duodenum, jejunum, proximal ileum, distal ileum, colon, rectum, or a biomarker thereof (e.g., a gut biomarker, cell surface marker, pH marker, chemical biomarker, extracellular matrix component, cellular secretion (e.g., mucus or component thereof), etc.). The chemical biomarker can include, for example, at least one of a gastric fluid component, a digestive fluid component, or an organ secretion. A pathogen, including the at least one targeted pathogen, can include a microbe, a viral agent (e.g., an enteric virus), a fungus, a parasite (e.g., a helminth or protozoan), or the like. A microbe can include a protozoan, intracellular parasite or microparasite, archaea, fungus or yeast, or bacteria. For example, a protozoan can include ameobae such as dysentery-causing Entamoeba histolytica, a cyclospora such as Cyclospora cayetanensis, or a parasite such as Giardia lamblia, a cryptosporidium, a microsporidium, or an isospora. For example, an intracellular parasite can include an obligate bacterial parasite such as a Mycobacterium or a protozoan such as a Cystoisosporim, a Cryptosporidium, Blastocystis hominis, or a Trypanosomatid like Leishmania. For example, archaea can include methanogenic archaea (e.g., such as Methanobrevibacter smithii, Methanosphaera stadtmanae, or members of the Methanomassiliicoccales order). For example, fungus can include a yeast such as Candida albicans or a parasitic fungi such as a Microsporidium. For example, bacteria can include a pathogenic bacteria such as members of the genera Escherichia, Klebsiella, Enterobacter, Serratia, and Citrobacter, Clostridium (e.g., C. difficile), Salmonella, Shigella or Campylobacter. For example, bacteria can include a nonpathogenic bacteria strain, e.g., a diet-related bacteria such as Bacteroides plebeius or a commensal microbe such as Lactobacillus acidophilus. In an embodiment, a pathogenic microbe can include a microbe not commonly found in a healthy gut, such as a microbe known to be associated with a disease. In an embodiment, a pathogenic microbe can include a displaced microbe, i.e., one that is commonly known to be resident and nonpathogenic in one part of the digestive system but is found in another part of the digestive system, e.g., the specific site 50 targeted by the composition, where its presence may be pathogenic or dysbiotic. For example, an anaerobic bacteria such as a Bacteroides, which is normally found in the colon, may be present in the small intestine, inducing dysbiosis or pathogenicity, and can induce a condition known as small intestinal bacterial overgrowth (SIBO). For example, intestinal dysbiosis in preterm infants that includes altered ratios of Proteobacteria, Firmicutes, and Bacteroidetes can precede infant necrotizing enterocolitis. In an embodiment, a pathogenic microbe can include a normally resident nonpathogenic microbe (i.e., a microbe commonly found at the specific site) that is present in abnormal quantities. For example, C. albicans normally found in very low levels in several places in the alimentary canal, including the mouth, throat, and colon, can become pathogenic upon overgrowth. For example, bacterial overgrowth syndrome (BOS) describes the clinical manifestation that occurs when the normally low number of bacteria that inhabit the stomach, duodenum, jejunum, or proximal ileum significantly increase or become overtaken by other pathogens.

The composition 100 includes a challenge agent 102 and a controlled release system 104. The challenge agent 102 is configured to elicit a measurable response by at least one targeted microbe at the specific site 50 in the gut 52. In embodiments, the measurable response is detectable in a biofluid of the individual subject. As used herein, the term “biofluid” can refer to a bodily gaseous emission (e.g., breath, breath condensate, respiratory gas, digestive gas, flatus), blood or component thereof, or a bodily excretion (e.g., saliva, urine, feces). For example, breath and breath condensate include gas phases, aerosol phases, and concentrated forms of expired fluids as bodily gaseous emissions, and may include respiratory gases (e.g., gases exhaled from the lungs, such as oxygen, carbon monoxide, carbon dioxide, nitrogen, etc., including those released from the bloodstream through alveolar gas exchange) and digestive gases (e.g., gases from the stomach, such as vapors expelled during belching). In embodiments, the measurable response detectable in the biofluid can include the presence of one or more of hydrogen, carbon dioxide, methane nitric oxide, nitrogen, ammonia, a volatile organic compound, or a taggant.

The challenge agent 102 can include, but is not limited to, at least one substrate 106 configured to interact with the at least one targeted microbe in a cell process. The substrate can include, for example, at least one saccharide 108 (e.g., a monosaccharide, a disaccharide, or a polysaccharide) or saccharide derivative such as an alcohol (e.g., sorbitol). In embodiments, the saccharide 108 can include one or more of fructose, lactose, glucose, sucrose, xylose, or lactulose. In embodiments, the type of saccharide 108 can depend on the specific site 50 of the gut 52 of interest and/or the type of target microbe. For example, a challenge agent that includes a saccharide such as glucose, xylose, or lactulose (e.g., which is not processed by human cells) can be targeted to the duodenum to detect the presence of anaerobic bacteria such as a Bacteroides. For example, a challenge agent that includes a saccharide such as fructose or lactose, which might be absorbed by human cells in the small intestine, can be targeted to colon (thereby bypassing the small intestine) to analyze the quantity of an anaerobic bacteria such as a Bacteroides. For example, a challenge agent that includes lactose can be targeted to the stomach to monitor levels of Lactobacillus acidophilus or Bifidobacterium lactis as part of a treatment for H. Pylori infection. For example, a challenge agent that includes lactose can be targeted to a specific microbe that produces lactase, such as Lactobacillus acidophilus, which might be monitored to determine if endogenous levels need to be supplemented by a probiotic treatment. The substrate 106 can include a taggant 110 to mark one or more components of the measurable response to identify such components are resultant from interactions with the challenge agent as opposed to other bodily functions. In embodiments, the taggant 110 can include one or more of a radioisotope, a fluorescent tag, a mass tag, or a chemical tag (e.g., a volatile organic compound). In embodiments, the challenge agent 102 includes multiple taggants 110, for example associated with the same or with different substrates. In embodiments, the substrate 106 configured to interact with the at least one targeted microbe in a cell process includes a metabolic substrate that interacts with the at least one targeted microbe in a metabolic cell process. For example, the challenge composition 100 can include glucose, which is utilized by some anaerobic microbes (e.g., commensal anaerobic microbes such as Bacteroides) in fermentation, a process that produces energy and releases as a byproduct hydrogen, which is absorbed into the bloodstream and exhaled via the lungs. For example, the challenge composition 100 can include a bile acid (e.g., glycocholate), which can be deconjugated by bacteria. In embodiments, the substrate 106 configured to interact with the at least one targeted microbe in a cell process includes an enzymatic substrate that interacts with the at least one targeted microbe in an enzymatic cell process. For example, the challenge composition 100 can include urea as a substrate for the enzyme urease; microbial urease (e.g., found in pathogenic microbes such as H. pylori and C. neoformans) processes the urea into ammonia and carbon dioxide, which are absorbed into the bloodstream and exhaled via the lungs. In embodiments, the substrate 106 configured to interact with the at least one targeted microbe in a cell process includes at least one taggant detectable in the biofluid (e.g., the measurable response detectable in the biofluid includes the taggant 110). The at least one taggant 110 can form at least a portion of the substrate 106, or can be included on a portion of the substrate 106. For example, the at least one taggant 110 can be integral to the substrate 106, such as urea having a radioactive carbon that upon metabolism is releasable as radioactive carbon dixoide, providing a measurable response (e.g., detectable as a radioactive signature). For example, the at least one taggant can be a releasable taggant (e.g., a volatile releasable tag) configured to be separated from the substrate 106, such as through cleavage by enzymatic or other cellular process of the at least one targeted microbe to induce the measurable response (e.g., detectable as a spectral signature). In embodiments, the cell process facilitated by interaction between the substrate 106 and the at least one targeted microbe provides a product that is a taggant or includes a taggant detectable in the biofluid. For example, the cell process can involve an enzymatic reaction that cleaves the taggant, an enzymatic reaction that adds the taggant to a final cell process product, or the like. In embodiments, the substrate 106 includes a methylated amine, such as trimethylamine (TMA), to interact with methyltransferases of archaea from the Methanomassiliicoccales order. In embodiments, the taggant 110 can be assimilated into the targeted microbe. For example, a stable isotope may be assimilated into a microbial molecule, e.g., DNA.

The specific site 50 of the gut 52 is a site of interest for determining an activity of microbes, presence of microbes, or the like. The specific site 50 of the gut 52 can be selected based on a variety of factors including, but not limited to, a likelihood of microbe activity; a particular microbiome of interest; a previous microbial exposure by the individual subject (e.g., an infection or an ingestion of a probiotic); a recommendation by a nutritionist, physician, or other healthcare professional (e.g., as part of a health examination); one or more symptoms experienced by the individual subject; one or more known or suspected health conditions of the individual subject; a geographical region inhabited, visited, or to be visited by the individual subject; a diet followed by the individual subject (e.g., vegetarian, vegan, regional cuisine, etc.); a desired outcome for the individual (e.g., weight loss, symptom relief, pathology treatment, etc.); or the like. For example, the stomach may be selected if the individual subject has been ingesting a probiotic (e.g., Lactobacillus acidophilus) as part of a treatment for H. Pylori infection of the stomach and wants to monitor levels of either the pathogen or the commensal bacteria. For example, if the individual subject experiences diarrhea, a healthcare professional may recommend a particular site of the intestinal tract be selected. For example, an intestinal site such as the colon can be selected if the individual subject is interested in monitoring levels of colonic flora, e.g., as part of a weight control program. For example, with regard to the geographical region or cuisine, enzymes that break down algal carbohydrates (e.g., β-porphyranases, certain agrases) can be found in the gut microbiota (Bacteroides plebeius) of Japanese sushi eaters, but generally are not found in humans from other geographical regions. In embodiments, the specific site 50 of the gut 52 includes, but is not limited to, the mouth, throat, esophagus, stomach, duodenum, jejunum, proximal ileum, distal ileum, colon, rectum, or combinations thereof.

In embodiments, the specific site 50 of the gut 52 includes a biomarker. For example, the biomarker can provide a targetable and/or recognizable element indicating the specific site 50. In embodiments, the biomarker can include, but is not limited to, a gut biomarker, a cell surface biomarker, a pH marker, a chemical biomarker (e.g., a gastric fluid component, a digestive fluid component, an organ secretion, etc.), an extracellular matrix component, a cellular secretion (a mucus, a mucus component, etc.), a biomarker of one or more resident microbes (e.g., a microbial surface molecule, a microbial functional biomarker, a microbial secreted molecule, a microbial extracellular substance, etc.). For example, a chemical biomarker can include a component of a digestive fluid present in a portion of the digestive tract, for example in the mouth a salivary component, in the stomach a gastric fluid component, or in the intestine an intestinal fluid component. Digestive fluid components can be specific to the site of production, functional in the conditions at the site such as the pH (while not functional or substantially less functional in conditions outside the site), and altered in a downstream portion of the digestive tract. A salivary fluid component can include, for example, an enzyme (e.g., lipase or amylase) or a protein (e.g., a mucin). Gastric fluid components can include, for example, an acidic component (e.g., hydrochloric acid), a specific enzymatic component (e.g., a protease such as pepsin or its precursor pepsinogen), a hormonal component (e.g., gastrin), or the like. An intestinal fluid component can include, for example, a compound found in the small intestine such as a hormone (e.g., secretin, cholecystokinin, etc.), a specific enzyme (e.g., maltase, lactase, erepsin, trypsin, etc.), acid-neutralizing agents (e.g., bicarbonates), bile, pancreatic juice, and the like. An intestinal fluid component can include, for example, a compound found in the large intestine or colon, such as a mucin or a bacterial-secreted compound. A component of a digestive fluid can be produced and secreted by an organ (e.g., the stomach, the pancreas, the liver, the small intestine) or an associated gland (such as a salivary gland, Brunner's gland, and other intestinal glands) or cells (e.g., in stomach (parietal cells, Chief cells, G cells, etc.), intestine (endocrine cells, Paneth cells, Goblet cells, etc.), pancreas (acinar cells, ductal cells, etc.). In one example, mucins produced by cells at different points of the digestive sentence can differ in specific structure and associated carbohydrate side chains depending on the site. In embodiments, the specific site 50 of the gut 52 includes a biomarker of one or more resident microbes (e.g., a microbe commonly found at a specific location of a body (e.g., in the gut) of a healthy individual). The biomarker can include, but is not limited to, a microbial extracellular substance. For example, microbial extracellular substances can include, but are not limited to, microbial extracellular polymeric substances (EPS) of microbial origin, a complex mixture of biopolymers comprising polysaccharides, proteins, nucleic acids, uronic acids, humic substances, lipids, microbial secretions, shed cell surface materials, cell lysates and adsorped environmental constituents. EPS make up the materials forming biofilm matrices, and serve in cell adhesion, immobilization, and spatial arrangement.

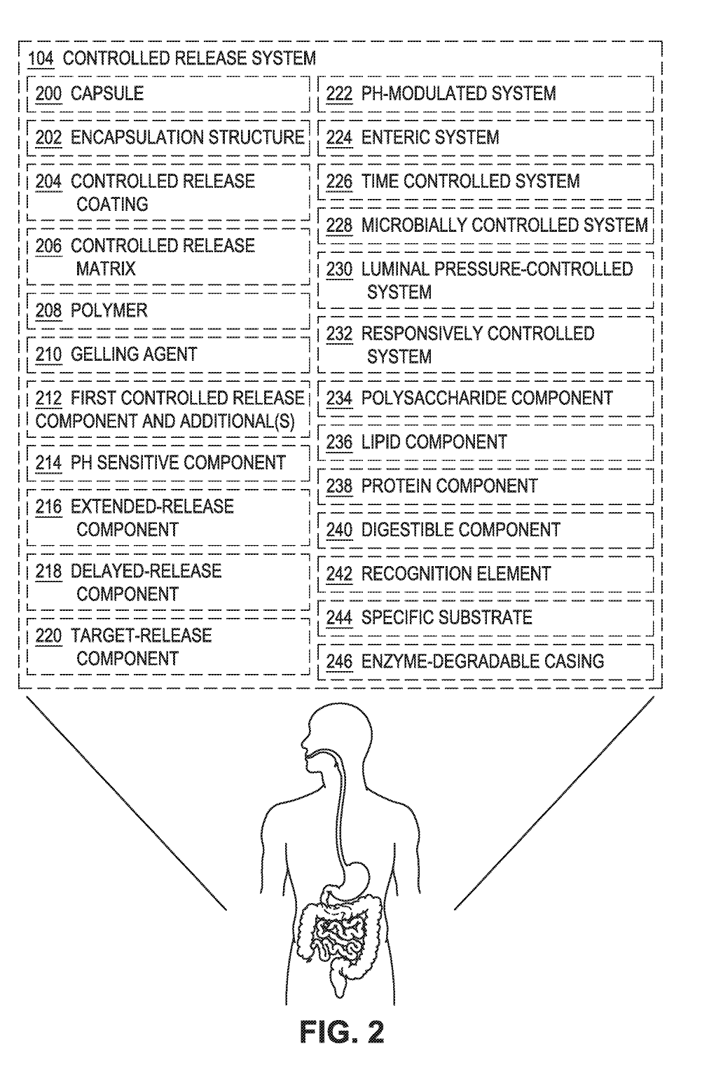

The controlled release system 104 of the composition 100 is configured to access the specific site 50 of the gut 52 to provide the challenge agent 102 the opportunity to elicit the measurable response by the at least one targeted microbe. In embodiments, the controlled release system 104 encompasses at least a portion of the challenge agent 102, providing protection and directed delivery of the challenge agent 102 and allowing delivery of the challenge agent 102 to the specific site 50 of the gut 52. In embodiments, shown in FIG. 2, the controlled release system can include, but is not limited to, a capsule 200, an encapsulation structure 202, a controlled release coating 204, a controlled release matrix 206, a polymer 208, a gelling agent 210, a first controlled release component and at least one additional controlled release component 212, at least one pH-sensitive component 214, an extended-release component 216, a delayed-release component 218, a target-release component 220, a pH-modulated system 222, an enteric system 224, a time controlled system 226, a microbially controlled system 228, a luminal pressure-controlled system 230, at least one responsively controlled system 232, a polysaccharide component 234, a lipid component 236, a protein component 238, a digestible component 240 that is digestable by one or more resident microbes at the specific site of the gut, a recognition element 242 to detect a biomarker present in the specific site of the gut, a specific substrate 244 that is specific for a resident microbial enzyme, an enzyme-degradable casing 246, or combinations thereof. In embodiments, the controlled release system 104 includes the capsule 200 or the encapsulation structure 202 to provide a material encapsulating or surrounding the entirety of the challenge agent 102 (e.g., the saccharide 108 or substrate 106, either of which may incorporate or be associated with the taggant 110).

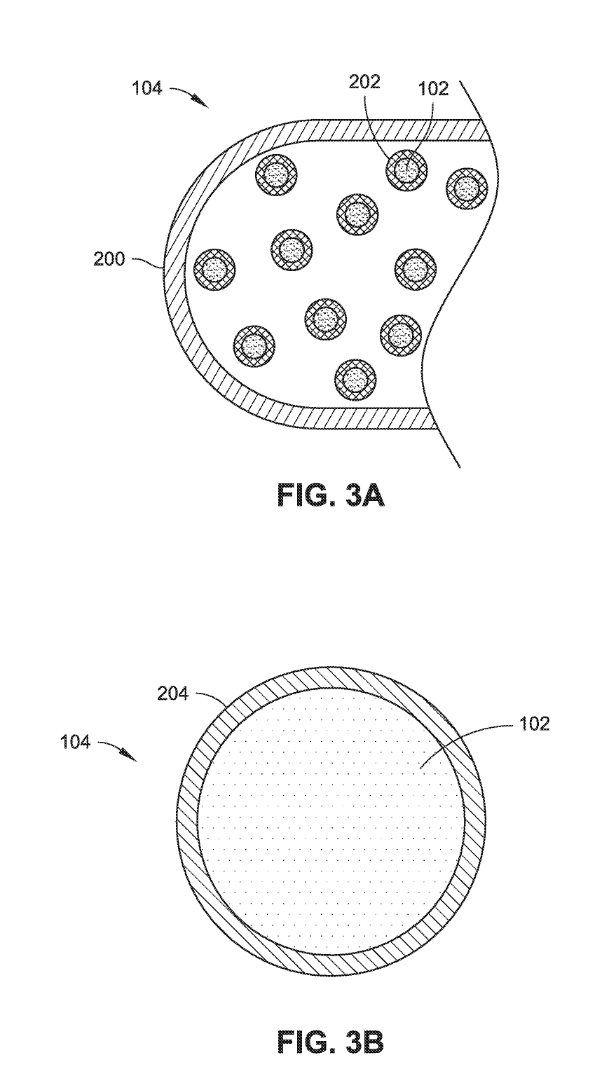

In embodiments, the controlled release system 104 can include one or more materials to encapsulate portions of the challenge agent 102, such as by dividing the challenge agent 102 into timed-release microparticles. For example, FIG. 3A shows the controlled release system 104 including a capsule 200 incorporating a plurality of encapsulation structures 202 to provide time-released microparticles of the challenge agent 102. In embodiments, the capsule 200 and the plurality of encapsulation structures 202 provide a layered system with sequential time-release properties. In embodiments, an example of which is shown in FIG. 3B, the controlled release system 104 includes at least one coating (e.g., controlled release coating 204), for example an enteric coating. In an embodiment, controlled release system 104 includes at least one matrix (e.g., controlled release matrix 206) incorporating both the challenge agent 102 and a controlled release material (e.g., one or more of the polymer 208, the gelling agent 210, the pH-sensitive component 214, the extended-release component 216, the delayed-release component 218, the target-release component 220, etc.).

In embodiments, the controlled release system 104 includes the polymer 208, such as a dissolution or diffusion polymer to carry the challenge agent 102 during transit to the specific site 50 of the gut 52, e.g., an upper gastric site. For example, the controlled release system 104 can include a diffusive membrane or matrix (e.g., a monolithic membrane) comprising a nonporous or microporous material (e.g., polymer or hydrogel). The polymer 208 can include, but is not limited to, cellulose derivatives, collagen, nylon, cyanoacrylates, polyethylene and derivatives, methacrylate derivatives, polyurethane, silicon, rubber, biodegradable polymers, poly(vinyl alcohol) (PVA), poly(acrylic acid) (e.g., CARBOPOL™), poly(ethylene oxide), poloxamers, pluronics, polymethacrylate (e.g. EUDRAGIT™), natural polymers (e.g., polysaccharides, proteins, cellulose derivatives including ethylcellulose, methylcellulose and their derivatives), or the like, or combinations thereof.

In embodiments, the controlled release system 104 includes one or more of a retentive component, such as a gastro-retentive component or intestine-retentive component, a swellable component, an extendable component, an unfoldable component, or a floating component, where such components allow the composition 100 to remain at or near the specific site 50 while the payload of the challenge agent 102 is delivered over time (e.g., released over time by one or more of the extended-release component 216, delayed-release component 218, target-release component 220, time controlled system 226, or other portion of the controlled release system 104). For example, a gastro-retentive controlled release system allows the composition 100 to remain in the stomach rather than passing into the intestine, thereby allowing the challenge agent 102 to be delivered over time to the stomach or to the duodenum. In embodiments, the controlled release system 104 includes a swellable component, such as a polymer, that swells or otherwise increases volume upon exposure to a particular pH (e.g., an acidic pH), which can include one or more of the pH-sensitive component 214, the pH-modulated system 222, etc. For example, the swellable component can include a polymer, such as a sodium starch glycolate or a sodium carboxymethylcellulose. In embodiments, the gastro-retentive component can include a swellable component to swell at acidic pH of the stomach. For example, a gastro-retentive component (e.g., one that is exposed after passing through an acidic environment) might swell at pH of 6 (e.g., in the duodenum) or at higher pH closer to 7.4 (e.g., in the ileum).

In embodiments, the controlled release system 104 includes at least one component of higher density than environmental fluids to which the composition 100 is introduced. For example, a sinking component having a density greater than that of the digestive fluids in the target gut site 50 (e.g., gastric juices of the stomach) can ensure the composition 100 is kept at the target gut site 50 (e.g., to the bottom of the stomach). In embodiments, the controlled release system 104 includes at least one floating component that is of lower density than environmental fluids to which the composition 100 is introduced, for example, a component having a density lower than that of gastric juices of the stomach, thereby allowing flotation of the composition 100 and retention within the stomach. For example, the controlled release system 104 can include a floating component having a lower density than that of the target gut site 50 through entrapment of air (e.g., hollow chambers) or by the incorporation of low density materials (e.g., fatty materials or oils, polypropylene foam powder, matrix forming polymers, drug powder, etc.). Low density materials can include, for example, hollow microspheres, microballoons, or microparticles based on low density foam powder, cross-linked beads (e.g., porous alginate beads), or the like. For example, the controlled release system 104 can include a non-effervescent floating system prepared from gel-forming or highly swellable cellulose-type hydrocolloids, polysaccharides, or matrix forming polymers like polyacrylate, polycarbonate, polystyrene and polymethacrylate. For example, the controlled release system 104 can include one or more hydrodynamically balanced systems such as gel-forming hydrocolloids meant to remain buoyant on the stomach content. In embodiments, the controlled release system 104 includes one or more unfoldable or extendable components that include a capsule having at least a portion made from biodegradable polymer, which, originally compressed, expands into a geometric form such as a tetrahedron or ring, thus increasing its size so that it cannot pass through a sphincter (e.g., the pyloric sphincter) until it has degraded, allowing its payload (e.g., the challenge agent 102) to be delivered to the stomach over time. In embodiments, the controlled release system 104 includes an effervescent system, for example, one or more gas-producing materials. For example, the effervescent system can include one or more swellable polymers (e.g., polysaccharides, chitosan, etc.) with one or more effervescent components (e.g., sodium bicarbonate, citric acid, tartaric acid, etc.) to generate carbon dioxide bubbles that promote floatation of the controlled release system 104, the challenge agent 102, or combinations thereof.

In embodiments, the controlled release system 104 includes at least one bioadhesive or mucoadhesive. A mucoadhesive can include a mucoadhesive polymer, which may bind through nonspecific, noncovalent interactions, or which may include functional groups (e.g., thiols) that increase binding (e.g., via hydrogen bonds), or which may include specific binding moieties (e.g., lectins) for cell or tissue surfaces. Polymers with mucoadhesive properties include polyvinylpyrrolidone (PVP), methylcellulose (MC), sodium carboxy methylcellulose (SCMC), and hydroxy propyl cellulose (HPC) . A bioadhesive or mucoadhesive can include materials that increase pH-dependent intestinal adhesion site specificity, and may aide in adhesion to the intestinal wall therefore retention in the specific site, e.g., small intestinal adhesion. In embodiments, the controlled release system 104 includes at least one adherent compound.

In embodiments, the controlled release system 104 includes one or more of the pH-modulated system 222, the enteric system 224, the time-controlled system 226, the microbially controlled system 228, or the luminal pressure-controlled system 230. For example, the controlled release system 104 can include at least one enteric component that protects the composition 100 while passing through the stomach. For example, the enteric system 224 of the controllable release system 104 can include one or more enteric polymers, e.g., as a coating. Examples of enteric polymers include, but are not limited to, polyvinyl acetate phthalate (PVAP) (COATERIC™), cellulose acetate trimellitate (CAT), hydroxypropyl methylcellulose phthalate (HPMCP), hydroxypropylmethylcellulose acetate succinate (HPMCAS), methacrylic acid copolymer Type A, B, or C (EUDRAGIT™), cellulose acetate phthalate (CAP) (AQUATERIC™), or a shellac. In embodiments, the controlled release system 104 includes at least one component that is degraded (e.g., enzymatically degraded, degraded by redox reaction, etc.) by a microbe or a secretion of a microbe that is normally resident at the specific site 50 of the gut 52. For example, the controlled release system 104 can include (e.g., in a capsule, coating, or matrix) a natural polymer such as a saccharide that is degradable by one or more microbial enzymes (e.g., amylase, chitosanase, pectinase, inulinase, xylanase, dextranase, galactomannanase, and the like) at the specific site 50, where such enzymes can be within the microbe or secreted by the microbe. For example, disaccharides (e.g., lactose or maltose), oligosaccharides (e.g., cellobiose, cyclodextrins, lactulose) and polysaccharides (e.g., chitosan, pectin, chondroitin sulphate, cyclodextrin, dextrans, guar gum, insulin, amylase and bean gum) have been identified for controlled release systems utilized in the colon. In embodiments, the controlled release system 104 includes at least one of a polymer or a hydrogel having azo bonds configured to undergo at least one of a redox reaction or an enzymatic reaction at the specific site 50 of the gut 52. For example, the controlled release system 104 can include at least one substrate specific for a resident microbial enzyme (e.g., a microbe present at the specific site 50). For example, the controlled release system 104 can include an enzyme-degradable casing.

In embodiments, the controlled release system 104 includes the luminal pressure-controlled system 230, which can include a component that is sensitive to a pressure occurring at a specific location of the digestive system. The luminal pressure-controlled system 230 can include, for example, a variable thickness capsule breakable by pressure associated with a body lumen. For example, the luminal pressure-controlled system 230 can include an ethylcellulose capsule having a thickness (e.g., 30-50 micrometers) that is breakable by the peristaltic pressure of the colon to supply access to the challenge agent 102 within the colon.

In embodiments, the controlled release system 104 includes the responsively controlled system 232. For example, the responsively controlled system 232 can include a component having a material configured for targeted release or responsive release of the challenge agent 102. For example, the controlled release system 104 can include the challenge agent 102 incorporated in a swellable hydrogel (e.g., hydroxypropyl methylcellulose) that swells in the presence of a fluid (e.g., a gastric juice) and upon a change in configuration of the hydrogel due to the swelling, delivers its payload. For example, the controlled release system 104 can include the challenge agent 102 incorporated in a responsive material such as a responsive (smart) hydrogel, the composition or structure of which is altered in response to a reaction. In one example, cleavage of polymer chains of a hydrogel via hydrolytic or enzymatic degradation can induce release of the payload substrate (e.g., the challenge agent 102). In another example, reactions occurring in the hydrogel to an external condition such as a change in pH can alter the structure of the hydrogel, thereby releasing the payload substrate. In yet another example, the binding of a ligand (e.g., in the environment of the specific site 50) to a specific recognition element incorporated in the responsive gel can induce an alteration in the structure of the hydrogel, thereby releasing the challenge agent 102. In embodiments, the responsively controlled system 232 includes an ion exchange material configured to release the challenge agent 102 upon exposure to a targeted ion within the gut 52 (e.g., an ion present at the specific site 50). In embodiments, the responsively controlled system 232 includes a passivelyr esponsive component. In embodiments, the responsively controlled system 232 includes an actively responsive component. In embodiments, the responsively controlled system 232 includes an electroresponsive component, a magnetic-responsive, an electrochemical component or the like. For example electro- or magnetic-responsive components might respond to an electric or magnetic charge that is on board the composition or provided by a separate entity such as a lumen traveling device or capsule endoscope.

In embodiments, the controlled release system 104 includes the polysaccharide component 234. The polysaccharide component 234 can include, but is not limited to, trehalose. In embodiments, the polysaccharide component 234 is digestible by one or more resident microbes at the specific site 50 of the gut 52 (e.g., a microbe commonly found at a specific location of a body (e.g., in the gut) of a healthy individual).

In embodiments, the controlled release system 104 includes the lipid component 236. The lipid component 236 can include, but is not limited to, one or more lipid micelles or liposomes. In embodiments, the lipid component 236 is digestible by one or more resident microbes at the specific site 50 of the gut 52.

In embodiments, the controlled release system 104 includes the protein component 238. In embodiments, the protein component 238 is digestible by one or more resident microbes at the specific site 50 of the gut 52.

In embodiments, the controlled release system 104 includes one or more hydrophobic components. For example, a hydrophobic controlled release system can include, but is not limited to, ethyl cellulose, liposomes, emulsions, encapsulations, microparticles, or combinations thereof. In embodiments, the controlled release system 104 includes one or more hydrophilic components. For example, a hydrophilic controlled release system can include, but is not limited to,

In embodiments, the controlled release system 104 includes the recognition element 242. The recognition element 242 can recognize the specific site 50 or regions of the gut 52 proximal the specific site 50 to provide the challenge agent 102 to the specific site. For example, the recognition element 242 is configured to recognize at least one biomarker at the specific site 52 in the gut 50, such as by including at least one of a binding agent, a ligand, an antibody or portions thereof, an aptamer, a molecularly imprinted gel, etc., or combinations thereof. In embodiments, the recognition element 242 is configured to recognize at least one biomarker at the specific site 52 in the gut 50, where the recognition element 242 is configured to recognize at least one of a cellular component or an extracellular matrix component.

In embodiments, the controlled release system 104 includes two or more controlled release components. In embodiments, the controlled release system 104 includes at least one of a first controlled release component and at least one second controlled release component. For example, the controlled release system 104 can include a controlled release component that protects the challenge agent 102 as it passes through at least a portion of the alimentary canal (e.g., stomach) yet allows controlled release at a later portion of the alimentary canal that is the specific location 50 of the gut 52 (e.g., the colon). In embodiments, the controlled release system 104 includes at least one second controlled delivery component that is the same type (e.g., pH-dependent component) as the first controlled delivery component. For example, the controlled release system 104 can include a pH-sensitive polymer coating as the first controlled delivery component surrounding a pH-sensitive matrix core as the second controlled delivery component. The pH-sensitive polymer protects the pH-sensitive matrix core at low pH of the stomach but dissolves at the higher pH of 6 in the duodenum. The pH-sensitive matrix core can incorporate the challenge agent 102 and a pH-dependent polymer that dissolves at the higher pH of 7.4 of the ileum. In embodiments, the controlled release system 104 includes a first pH-sensitive component configured to access a first gut site (e.g., the stomach) and at least one additional pH-sensitive component configured to access at least one additional gut site (e.g., the colon).

In embodiments, the composition 100 includes at least one second controlled delivery component that is different than the at least one first controlled delivery component. For example, the controlled release system 104 can include an outer layer of an enteric polymer coating (e.g., cellulose acetate phthalate) surrounding a core incorporating the challenge agent and a controlled release matrix of a pH-independent polymer (e.g., ethylcellulose), which is resistant to neutral or alkaline aqueous medium, and microcrystalline cellulose (MCC), which is digestible by specific enzymes present in the colon. For example, the composition 100 can include a center core comprising the challenge agent 102 surrounded by a coating layer of chitosan and a top coat of an enteric polymer such as hydroxypropyl methyl cellulose acetate succinate (HPMCAS) or hydroxypropyl methyl cellulose hexahydrophthalate. The enteric polymer protects the composition 100 through the stomach and allows the core to reach the colon, where the chitosan coating is degraded by a chitosanase, thereby delivering the payload challenge agent 102 to the specific location 52 of the colon.

In embodiments, the controlled release system 104 can include two or more components in an arrangement. For example the controlled release system 104 can include two or more layers. For example, the controlled release system 104 can include two or more sections.

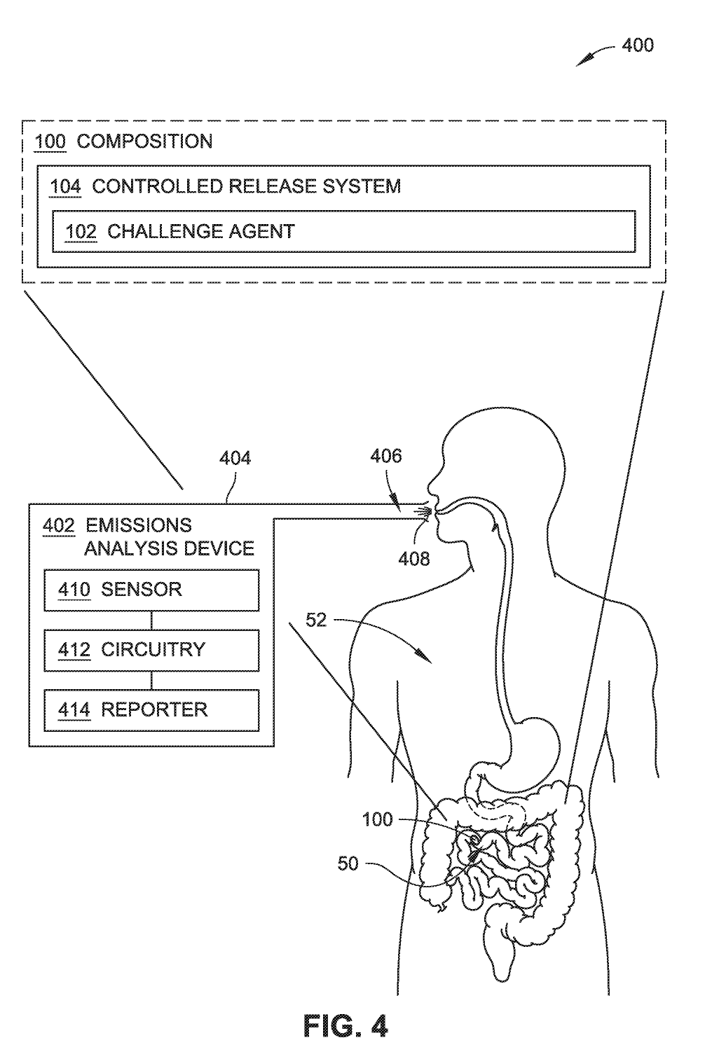

Upon release or other delivery of the challenge agent 102 by the controlled release composition 104, the challenge agent 102 is available to elicit a measurable response by at least one targeted microbe at the specific site 50 in the gut 52. In embodiments, the measurable response can be detected by a sensor, such as by sensing the presence of at least one analyte in the biofluid of the individual. The sensor can be incorporated into, or can include, an emissions analysis device. Referring to FIG. 4, a system 400 is shown including the composition 100 and an emissions analysis device 402 having a body structure 404 defining an orifice 406 configured to receive a biofluid 408 from the individual, a sensor 410, control circuitry 412, and a reporter 414. While the emissions analysis device 402 is shown receiving an oral emission from the individual (e.g., a breathalyzer device configured to receive a bodily gaseous emission), the emissions analysis device 402 can also include, but is not limited to, an intraoral device, an endoluminal device, or combinations thereof to receive a biofluid in vivo. For example, the emissions analysis device 402 can be or can include an endoluminal device or an intraoral device configured for delivery to a site in the alimentary canal. In embodiments, the emissions analysis device 402 can be delivered by oral administration (e.g., placing in an oral cavity), by ingestion (e.g., ingestion of a capsule endoscope or lumen traveling device), by endoscope (emplacement near or at the specific site of the gut via an orally introduced or rectally introduced endoscope), or by implantation. For example, the emissions analysis device 402 can include a capsule endoscope or a lumen traveling device introduced into the alimentary canal to receive the biofluid in vivo. In embodiments, the emissions analysis device 402 can be configured to receive colonic gaseous emissions or flatus.

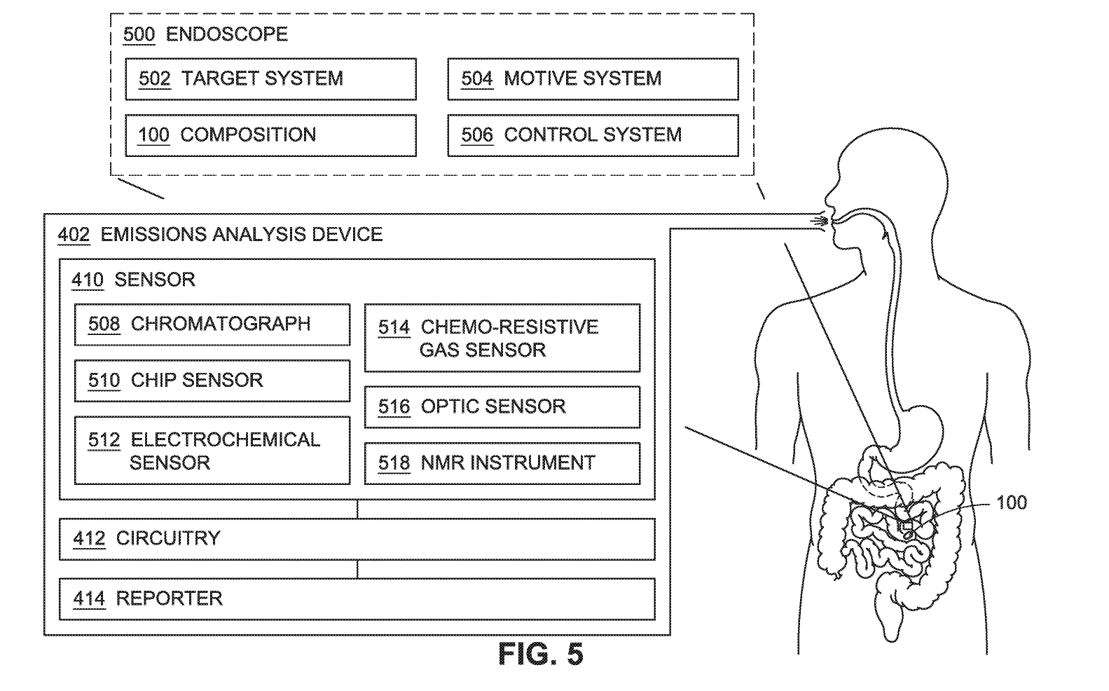

In embodiments, shown in FIG. 5, the composition 100 is delivered to the specific site 50 of the gut 52 by an endoscope 500 (e.g., capsule or pill endoscope, intraluminal endoscope, etc.) or other intraluminal device (e.g., intraoral device). For example, the endoscope can include a target system 502, a motive system 504, and a control system 506 to facilitate intraluminal operation. The target system 502 can detect the specific site 50 of the gut 52, and can include, but is not limited to a recognition element or optics to provide imaging for identifying a site along the alimentary canal (e.g., the specific site 50 or other region of the alimentary canal), for detecting a tag delivered by the endoscope 500 or the composition 100, or combinations thereof. Upon detection of the specific site 50, the endoscope 500 can release the composition 100 to challenge target microbes at the specific site 50. The target system 502 can also include control programming for facilitating operations of the endoscope 500. The motive system 504 and the control system 506 can provide controlled travel of the endoscope 500 in the lumen. For example, the control system 514 can include one or more steering devices to alter a path of travel of the endoscope 500 through the lumen, whereas the motive system 504 induces one or more forces to propel the endoscope 500 through the lumen. The target system 502, the motive system 504, and the control system 506 can be located ex vivo (e.g., when endoscope 500 is an intraluminal endoscope) or in vivo (e.g., when endoscope 500 is a capsule endoscope). In embodiments, the composition 100 is delivered to the specific site 50 of the gut 52 via oral administration. For example, the composition 100 can be administered as a solid (e.g., capsules, tablet, powder), a fluid, or a liquid or can be solution or suspension, emulsion, gel, or the like. For example, the composition can be ingested in food or drink. In embodiments, the composition 100 is delivered to the specific site 50 of the gut 52 via rectal administration, for example in suppository or enema. In embodiments, the composition 100 is delivered to the specific site 50 of the gut 52 via an injection, such as through a transcutaneous injection via needle, microneedle, or the like. In embodiments, the composition 100 is delivered to the specific site 50 of the gut 52 via an implant positioned at least partially within the user, such as a surgical implant configured to release compositions (e.g., from a reservoir on or in the implant).

The emissions analysis device 402 can include a chromatograph 508, a chip sensor 510, an electrochemical sensor 512, a chemo-resistive gas sensor 514, an optic sensor 516, a nuclear magnetic reasonance (NMR) instrument 518, or combinations thereof to facilitate operation of the emissions analysis device 402, such as by detecting and/or analyzing the biofluid 408 (e.g., exhalant) received from the individual subject. For example, the sensor 410 can include the chromatograph 508, the chip sensor 510, the electrochemical sensor 512, the chemo-resistive gas sensor 514, the optic sensor 516, the NMR instrument 518, or combinations thereof.

In embodiments, the emission analysis device 402 is an ex vivo device. In embodiments, the emission analysis device 402 is a portable device configured to be transported by the individual or other individual (e.g., a breathalyzer device configured to receive a bodily gaseous emission). In embodiments, the emission analysis device 402 is a hand-held device having dimensions suitable for transporting and manipulating by hand. In embodiments, the NMR instrument 518 includes a handheld NMR device.

Referring again to FIG. 4, the emissions analysis device 402 is shown having the sensor 410, the circuitry 412, and the reporter 414. In general, the sensor 410 is operably coupled to the orifice 406 and configured to detect at least one analyte from the biofluid 408 (e.g., a bodily gaseous emission) and generate one or more sense signals associated with the measurable response elicited by the challenge agent 102 of the composition 100 administered to the individual. For example, the measurable response can include, but is not limited to, one or more of hydrogen, carbon dioxide, nitric oxide, nitrogen, a volatile organic compound, or a taggant. The sensor 410 can include a single sensor or a plurality of sensors, such as a sensor array. For example, the sensor 410 can include one or more of an optical sensor, an acoustic sensor, an electromagnetic sensor, a magnetic sensor, an electrophoretic sensor, an electrochemical sensor, a biochemical sensor, a microfluidic sensor, a magnetic resonance sensor, a piezoelectric sensor, a surface plasmon resonance sensor, an optical microsensor array, a surface enhanced raman spectrometer (SERS), a laser, an ion flow tube, a metal oxide sensor (MOS), an infrared spectrophotometer, an acoustic wave sensor, a colorimetric tube, a conductive-polymer gas sensor, a chemoresistor, a selective resonance sensor, a gas chromatograph, a mass spectrophotometer, or combinations thereof. In embodiments, the sensor 410 is a chip-based sensor including one or more of an “electric nose” sensor (e-nose sensor), a cantilever-based sensor, surface acoustic wave (SAW) sensor, a semiconductor metal oxide (SMO)-based chemiresistive sensor, or combinations thereof. In embodiments, the sensor 410 includes one or more of a portable hydrogen sensor (e.g., a micro H2 sensor, a GASTROPLUS™ sensor, etc.), a fuel cell sensor, or combinations thereof. In embodiments, the sensor 410 includes a solid state gas sensor, including but not limited to, a nanostructured metal oxide-based chemo-resistive gas sensor, a Micro Electro Mechanical System (MEMS) sensor, or the like. In embodiments, the sensor 410 includes nanotechnology-based sensing components. For example, the sensor 410 can include one or more of an array-based sensor having nanoparticles (e.g., providing ligands to interact with analytes in the biofluid 408, such as VOCs), modified gold nanoparticles (GNPs), carbon nanostructures, microporous metal organic frameworks (MMOF), or the like. In embodiments, the sensor 410 is configured to measure multiple analytes in a gaseous bodily emission (e.g., hydrogen, carbon monoxide, hydrogen sulfide, ammonia, and ethanol) by incorporating a sensor array having a plurality of electrochemical sensors (e.g., for sensing hydrogen, carbon monoxide, hydrogen sulfide and ethanol), a ceramic sensor (e.g., to measure total volatiles), and a dye-based optical sensor (e.g., for sensing ammonia). In embodiments, the sensor 410 includes a flexible substrate supporting molecularly modified gold nanoparticles, where a bending state of the GNP-based flexible substrate provides unique nanoparticle spatial organization, permitting alterable interactions between GNP ligands and VOCs based on the bending states.

The circuitry 412 is operably coupled to the sensor 410 and is configured to receive the one or more sense signals generated by the sensor 410 for processing and analysis (e.g., to determine a particular microbiome distribution, to identify the presence or absence of a particular microbe, such as the target microbe, etc.). The reporter 414 is coupled to the circuitry 412 and is configured to generate one or more communication signals associated with the microbe targeted by the composition 100 (i.e., the targeted microbe), where such communication signals can be displayed or transmitted to provide information about the targeted microbe, or to provide additional analysis of the information obtained and/or analyzed by the emissions analysis device 402.

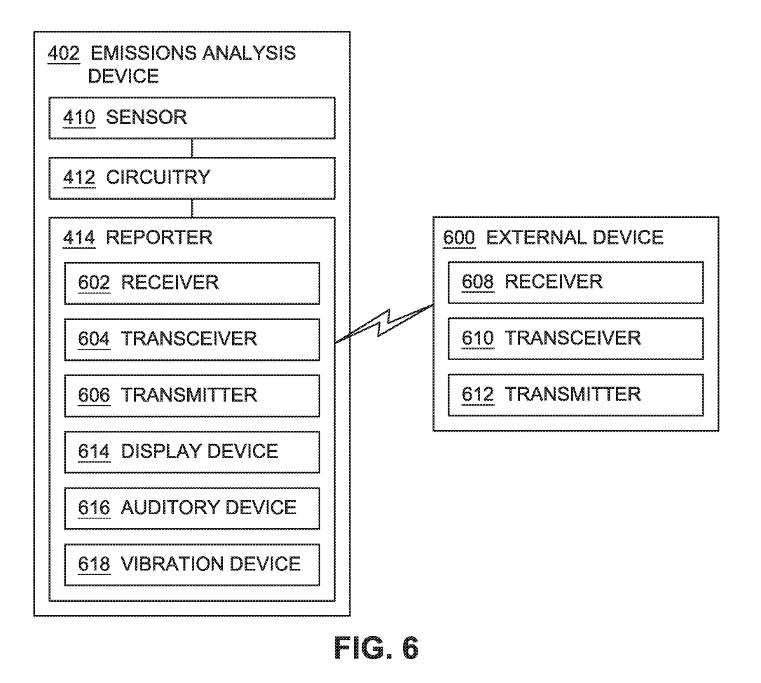

The circuitry 412 includes components to process the one or more sense signals from the sensor 410 and to provide instruction to one or more components of the system 400, such as the reporter 414. For example, the circuitry 412 can include, or comprise a portion of, a microprocessor, a central processing unit (CPU), a digital signal processor (DSP), an application-specific integrated circuit (ASIC), a field programmable gate entry (FPGA), or the like, or any combinations thereof, and can include discrete digital or analog circuit elements or electronics, or combinations thereof. In an embodiment, the circuitry 412 includes one or more ASICs having a plurality of predefined logic components. In an embodiment, the circuitry 412 includes one or more FPGAs having a plurality of programmable logic commands. The computer memory device can be integrated with the system 400, can be associated with an external device and accessible by the system 400 through wireless or wired communication protocols, or a combination thereof. For example, reference data (e.g., target microbial reference data) can be stored by computer memory coupled to or supported by the body structure 404 of the system 400, can be accessible by the circuitry 412 via wireless means, can be available to the circuitry 412 through another method, such as through a remote network, a cloud network, and so forth, or combinations thereof. In embodiments, an example of which is shown in FIG. 6, the circuitry 412 is operably coupled to an external device 600 via one or more of a receiver 602, a transceiver 604, or a transmitter 606 (e.g., portions of an antenna or broadcast system). For example, the reporter 414 can include, or is operably coupled to, the receiver 602 or transceiver 604 (e.g., antenna, etc.), or combinations thereof, to receive information (e.g., control programming, reference information, etc.) or communications to facilitate operation or control of the system 400 through wireless or wired communication protocols. For example, the receiver 602 or transceiver 604 can receive one or more communication signals from the external device 600 associated with but not limited to, control programming, reference data, a query (e.g., a query to transmit information from the system 400 to the external device 600, a query to determine an operational state of the sensor 410, a query to determine a status of a microbiome within the gut 52 of the individual, a query to determine a composition of the microbiome at the specific site 50, etc.), or combinations thereof. In embodiments, the reporter 414 can additionally or alternatively include the transmitter 606 or transceiver 604 (e.g., antenna, etc.) to send information amongst components of the system 400 or to components external the system, such as to communicate with the external device 600 (e.g., to communication with a healthcare professional, a guardian or representative of the individual subject, an insurance company representative, or the like). Such communication can include, for example, indications that the circuitry is accessing one or more databases or memory devices storing reference or programming data, computational protocols, system updates, information or data transfer, or the like. The external device 600 can include one or more of a receiver 608, a transceiver 610, or a transmitter 612 to facilitate communications with the components of the system 400. The external device 600 can include but is not limited to, a communication device or electronic equipment, such as one or more of a mobile communication device, mobile platform, or a computer system including, but not limited to, one or more mobile computing devices (e.g., hand-held portable computers, Personal Digital Assistants (PDAs), wearable healthcare platform, laptop computers, netbook computers, tablet computers, or so forth), devices supporting network functionality, mobile telephone devices (e.g., cellular telephones and smartphones), devices that include functionalities associated with smartphones and tablet computers (e.g., phablets), wearable or portable devices (e.g., including sensors positioned on the individual subject, sensors positioned remotely from the individual subject, sensors positioned on different individual subjects, etc.), portable game devices, portable media players, multimedia devices, kiosks, augmented or virtual reality (VR) systems (e.g., VR headsets, VR immersive experience systems, etc.) satellite navigation devices (e.g., Global Positioning System (GPS) navigation devices), e-book reader devices (eReaders), Smart Television (TV) devices, surface computing devices (e.g., table top computers), Personal Computer (PC) devices, and other devices that employ touch-based human interfaces. The system 400 and the external device 600 can communicate respective each other (e.g., send and receive communication signals) via the receivers 602, 608, the transceivers 604, 610, and the transmitters 606, 612, such as through one or more connected and wireless communication mechanisms including, but not limited to acoustic communication signals, optical communication signals, radio communication signals, infrared communication signals, ultrasonic communication signals, and the like. In an embodiment, the system 400 can utilize communications from the external device 600 as an operational indicator (e.g., when to begin sensing via the sensor 410, when to operate the reporter 414, etc.).

In embodiments, the reporter includes one or more of a display device 614, an auditory device 616, or a vibration device 618 to provide alerts or other information associated with the at least one targeted microbe targeted by the composition 100. For example, the display device 614 can be configured to provide a visual indication pertaining to the at least one targeted microbe, the auditory device 616 can be configured to provide an auditory indication pertaining to the at least one targeted microbe, and the vibration device 618 can be configured to provide a vibratory indication pertaining to the at least one targeted microbe.

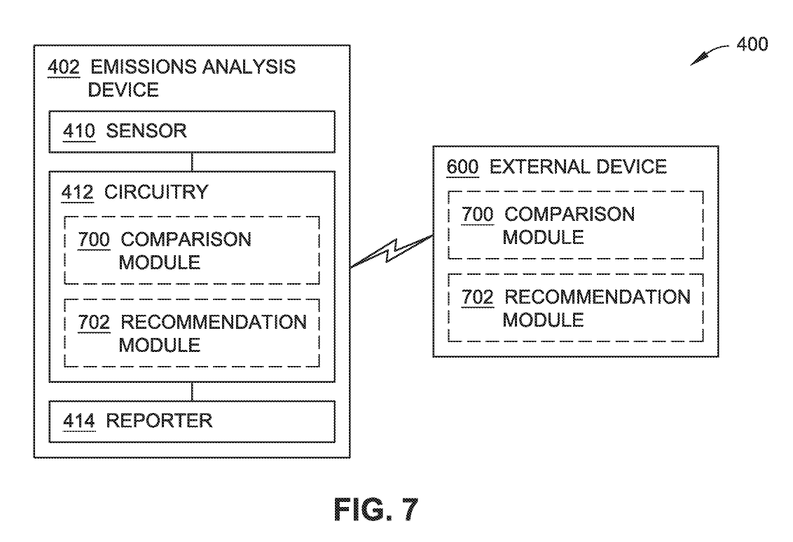

In embodiments, an example of which is shown in FIG. 7, the system 400 can include one or more of a comparison module 700 configured to compare information obtained by the sensor 410 to reference data for analysis of the targeted microbe and a recommendation module 702 configured to generate a recommendation for the individual, for a healthcare professional (including a private or public health official), for a third party (e.g., an insurance company representative, a guardian or representative for the individual, a researcher, etc.), or the like, responsive to operation of the system 400. For example, one or more of the circuitry 412 of the emissions analysis device 402 and the external device 600 can include or incorporate one or more of the comparison module 700 and the recommendation module 702. The comparison module 700 compares the sense signals generated by the sensor 410 to reference data, where the reference data can be made available to the circuitry 412 or the external device 600 through one or more of access of a memory device (via physical access, wirelessly access, etc.) by the circuitry 412 or the external device 600, through communications from the circuitry 412 (e.g., when the external device 600 includes or accesses the comparison module 700), through communications from the external device 600 (e.g., when the circuitry 412 includes or accesses the comparison module 700), through upload via a user interface incorporated by one or more of the emissions analysis device 402 and the external device 600, or combinations thereof. The reference data can include, but is not limited to, microbe types, microbiome distributions, correlations between microbe type and detectable analyte, correlations between microbiome distribution and detectable analyte, correlations between microbe type and a risk of disease or physiological disorder (e.g., infection or microbial overgrowth, inflammation (e.g., irritable bowel syndrome, arthritis), autoimmunity (e.g., Crohn's disease, autoimmune arthritis), metabolic disorders (e.g., obesity), cardiac disorders, circulatory disorders, mental disorders, emotional disorders, etc.), correlations between microbiome distribution and a risk of disease or physiological disorder (e.g., infection or microbial overgrowth, inflammation, (e.g., irritable bowel syndrome, arthritis), autoimmunity (e.g., Crohn's disease, autoimmune arthritis) metabolic disorders (e.g., obesity), cardiac disorders, circulatory disorders, mental disorders, emotional disorders, etc.), correlations between microbe type and a physiological benefit (e.g., treatment or prevention of pathogenic colonization, metabolic benefits, etc.), correlations between microbiome distribution and a physiological benefit (e.g., treatment or prevention of pathogenic colonization, metabolic benefits, etc.), correlations between microbe type and drug uptake or efficacy, correlations between microbiome distribution and drug uptake or efficacy, correlations between microbe type and population data, correlations between microbiome distribution and population data, or the like. Based on the particular reference data utilized, the comparison module 700 can compare the reference data to the output of the sensor 410 when detecting the measurable response elicited by the target microbe responsive to interaction with the challenge agent 102 to make a determination as to a type of microbe detected, a microbiome distribution detected, an amount of analyte detected and the correlated microbe type, an amount of analyte detected and the correlated microbiome distribution, a risk of disease or physiological disorder, or combinations thereof. In embodiments, the comparison module 700 makes comparisons between the output of the sensor 410 and the reference data covering a period of time (e.g., every minute, every hour, every day, every week, or other schedule of comparison on a periodic basis). For example, by making comparisons over a period of time, the comparison module 700 can make time-based determinations, such determining whether the individual is experiencing the onset of a disease or physiological disorder (e.g., one or more of a pathogenic infection, dysbiosis, inappropriate growth, an inflammatory disorder, a hyperimmune disorder, or an autoimmune disorder). In embodiments, the circuitry 412 is configured to provide a communication to a healthcare professional responsive to comparison of the output of the sensor 410 to the reference data. The communication can be provided via the reporter 414 on a real-time basis, on a periodic basis, on a one-time basis, etc. For example, the circuitry 412 can open a line of communication with the healthcare professional via the reporter 414 to permit the individual subject to speak or otherwise communicate when it is determined that the target microbe or microbial population of the specific site 52 is a health concern or complex matter for discussion. In embodiments, the circuitry 412 is configured to communicate with a source of products via an external network (e.g., an external health network supported by the external device 600) to order at least one recommended product for use by the individual. The recommended product can include, but is not limited to, a food or drink, a probiotic, a prebiotic, a specific microbe, a nutrient or nutraceutical, a therapeutic treatment (e.g., antimicrobial treatment, anti-inflammatory treatment, etc.), a medication, or the like, or combinations thereof.

The output of the comparison module 700 can be used as a factor for analysis by the recommendation module 702 to generate one or more recommendations to the individual subject or third party based on operation of the system 400. For example, one or more of the circuitry 412 of the emissions analysis device 402 and the external device 600 can include or incorporate the recommendation module 702 to provide generate a recommendation responsive to comparison of the one or more sense signals from the from sensor 410 to the reference data. For example, if the comparison module 700 determines that the individual subject is at risk for a disease or physiological disorder based on the type of microbe or microbiome distribution detected, the recommendation module 702 can provide recommendations to the individual subject or third party to mitigate or reduce the risk factor, such as a recommendation to alter a microbial population of the specific site 52 of the gut 50. The recommendations generated by the recommendation module 702 can include, but are not limited to, a recommendation to increase a particular microbe population, a recommendation to decrease a particular microbe population, a recommendation to communicate with a healthcare professional or public health official, a recommendation to change a diet, a recommendation to introduce a food or drink, a recommendation to introduce a probiotic, a recommendation to introduce a prebiotic, a recommendation to introduce a specific microbe (e.g., a commercially available microbe, a stored microbe (e.g., pre-operative sample), a microbe collected from a specific individual (e.g., a family member or a celebrity) or type of individual (e.g., having a specific trait such as high metabolism), a genetically engineered microbe, or the like), a recommendation for a fecal transplant (e.g., from a user's frozen sample, a family member, etc., such as by introduction of a washed sample via endoscope), a recommendation to introduce a nutrient or nutraceutical, a recommendation to undergo a therapeutic treatment (e.g., antimicrobial treatment, anti-inflammatory treatment, etc.), a recommendation to alter a therapeutic treatment, a recommendation to increase physical activity, or the like, or combinations thereof. The recommendation to change the diet of the individual can include recommendations to ingest a particular substance, such as one or more of a saccharide, a lipid, a protein, a nutrient, or the like. For example, the comparison module 700 can compare the sense signals from the sensor to the reference data that includes microbe or microbiome data from multiple users or a user population at a single time point or over time, and make a recommendation via the recommendation module 702. Such a recommendation, for instance, can include information regarding the health of a population (e.g., outbreak of a pathogen) or the response of a population (e.g., response to a shared food such as to identify a pathogen or allergen, or response to a shared situation).

In embodiments, the emissions analysis device 402 is incorporated in a kiosk. In embodiments, the external device 600 includes a kiosk. For example, the individual subject can provide the biofluid 408 to the orifice 406 at the kiosk, where the kiosk or the emissions analysis device 402 can include the recommendation module 702 to generate a recommendation following analysis of the biofluid 408 by the sensor 410 and the comparison module 700. In embodiments, the emissions analysis device 402 is communicatively coupled with a kiosk to transmit information (e.g., via wireless or wired communication protocols) to the kiosk, such as via the reporter 414, described with reference to FIG. 6. In embodiments, the kiosk is configured to provide at least one product for consumption to aid in altering a microbial distribution within the gut 52 responsive to communications from the recommendation module 702. For example, the recommendation module 702 can generate a communication regarding a recommendation for the individual to consume a product based on a microbial distribution present in the gut 52 (e.g., as determined by the comparison module 700. The product can include, but is not limited to, a food or drink, a probiotic, a prebiotic, a specific microbe, a nutrient or nutraceutical, a therapeutic treatment (e.g., antimicrobial treatment, anti-inflammatory treatment, etc.), a medication, or the like, or combinations thereof. The kiosk can dispense the product to the individual subject, can transmit a retail order to procure the product, or combinations thereof. Alternatively or additionally, the external device 600 can transmit a retail order to produce the product.

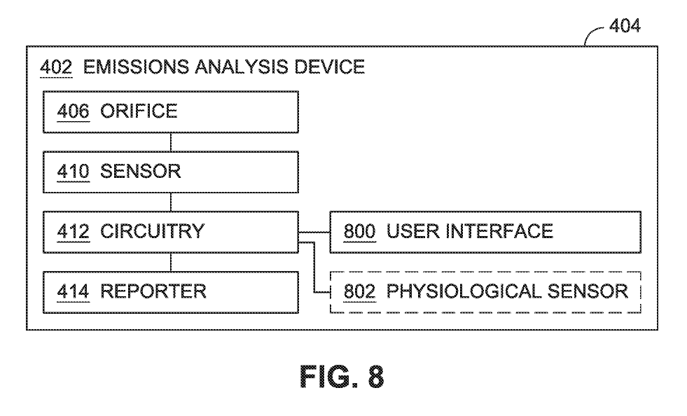

In embodiments, an example of which is shown in FIG. 8, the emissions analysis device 402 includes a user interface 800. The user interface 800 can be operably coupled to the circuitry 412 to facilitate operations of the system 400. For example, the user interface 800 can receive a user input from an individual subject, display an output of the one or more communication signals associated with the at least one targeted microbe, display an output associated with an operation of the emissions analysis device, or combinations thereof. The output from the user interface can include, but is not limited to, visual output (e.g., text, graphics, etc.), audio output, tactile output, or combinations thereof. For example, the user interface 800 can generate one or more communication signals for display by the user interface 800. The communication signals for display can include, for example, a request for user input regarding an operation state of the emissions analysis device 402 (e.g., whether the individual is ready to provide the biofluid to the orifice 406). The user interface 800 can include, but is not limited to, a voice input interface, a graphical user interface (GUI), a touchscreen assembly (e.g., a capacitive touch screen), a liquid crystal display (LCD), a light-emitting diode (LED) display, or projection-based display, or combinations thereof.

In embodiments, the emissions analysis device 402 is structured and dimensioned as a hand-held unit incorporating at least each of the orifice 406, the sensor 410, the circuitry 412, the reporter 414, the comparison module 700, the recommendation module 702, and the user interface 800. The individual can introduce the composition 100 (e.g., via ingestion), whereby the controlled release system 104 facilitates distribution of the challenge agent 102 at the specific site 50 of the gut 52, as described herein. In embodiments, the individual can enter a time of ingestion of the composition 100 into the user interface 800, and the circuitry 412 can calculate an expected time of eliciting the measurable response by the target microbe responsive to interaction with the challenge agent 102. For example, an orally administered composition 100 takes about three hours to travel through the length of the small intestine to the beginning of the colon. Depending on the specific site 50 of interest, the time from oral administration of the composition 100 to the time of eliciting the measurable response can be greater than three hours (e.g., for sites 50 at and following the colon) or less than three hours (for sites 50 before the colon). The circuitry 412 can then instruct the user interface 800 to prompt the individual (e.g., via display on the user interface 800) to provide a biofluid sample to the orifice 406 (e.g., via exhaled breath) for analysis by the sensor 410 and circuitry 412, via the comparison module 700 and the recommendation module 702. Following analysis, the recommendation module 702 can generate a recommendation for display on the user interface 800, for transmission to a third party (e.g., via the reporter 414), for the circuitry 412 to order a product or establish communications with a healthcare professional, or combinations thereof, or the recommendation module 702 can recommend that the individual wait a period of time before providing another sample of biofluid to the emissions analysis device 402 for subsequent analysis (e.g., if insufficient analytes are detected, if the analysis protocol calls for multiple analyses, or the like).

In embodiments, the system 400 includes at least one physiological sensor. The physiological sensor can include a heart rate sensor, a respiratory sensor, a thermal sensor, a blood pressure sensor, a hydration sensor, a chemical sensor, an oximetry sensor, a pressure sensor, or the like, or combinations thereof. The physiological sensor can provide information about a user through contact with the body of the user or proximity to the skin of the user. For example, shown in FIG. 8, a physiological sensor 802 can be housed in the emissions analysis device 402 (e.g., a breathalyzer) that physically touches the skin (e.g., a hand holding the breathalyzer) or the mouth of the subject and can include sensors and circuitry to detect a heart rate, a temperature, a respiratory rate, skin hydration, pulse oximetry, or the like, or combinations thereof. For example, the physiological sensor can be housed in an endoluminal device used in the delivery of the challenge agent or in an endoluminal emissions analysis device and can include physiological sensors such as a pressure sensor or temperature sensor that senses the luminal pressure or the luminal temperature. In an embodiment, the system 400 can report information from the physiological sensor to an external device (e.g., external device 600). For example, the physiological sensor can communicate with the mobile communication device, the mobile platform, the mobile healthcare platform, the kiosk, etc. via one or more communication protocols (e.g., wired communication protocol, wireless communication protocol, etc.). In an embodiment, the physiological sensor is housed in the external device 600. For example, the physiological sensor can be housed in the mobile communication device, the mobile platform, the mobile healthcare platform, the kiosk, etc. In embodiments, the system 400 utilizes sense signals from the physiological sensor (e.g., physiological sensor 802, a remote physiological sensor, such as a physiological sensor on an endoluminal device, etc.) as a trigger to determine whether to recommend to the individual subject that the challenge composition 100 be introduced. The sense signals generated by the physiological sensor can be analyzed via the comparison module 700 to determine whether the sense signals are indicative of a condition for which testing with the challenge composition 100 would be recommended (e.g., via the recommendation module 702). For example, if the subject is undergoing monitoring for a known health condition, changes in the sense signals from the physiological sensor over time, or as compared to a previous condition, previous sense signals, or reference sense signals, as compared by the comparison module 700 can be a trigger for the recommendation module 702 to recommend introducing the challenge composition 100 to determine a cause of the change, such as where the change in sense signals indicates that a health parameter of the individual could be abnormal. For example, if the subject is being treated for an infection and the physiological sensor generates sense signals indicating an increase in body temperature of the subject, the recommendation module 702 can generate a recommendation to introduce the challenge composition 100 to test for pathogen regrowth. For example, if the physiological sensor generates sense signals indicating an increase in weight of the individual subject (or portion thereof), the recommendation module 702 can generate a recommendation to introduce the challenge composition 100 to determine if an inflammation is present or changing. In embodiments, the system 400 introduces a survey to the individual subject via the user interface 800 to provide information to, and receive information from, the individual subject associated with one or more of diet, exercise, or sleep conditions. For example, input received from the individual subject via the user interface 800 can be analyzed by the comparison module 700 or the recommendation module 702, or combinations thereof, to determine whether to recommend introduction of the challenge composition 100, to determine whether to provide a recommendation to increase a particular microbe population, a recommendation to decrease a particular microbe population, a recommendation to communicate with a healthcare professional, a recommendation to change a diet, a recommendation to introduce a food or drink, a recommendation to introduce a probiotic, a recommendation to introduce a prebiotic, a recommendation to introduce a specific microbe, a recommendation to introduce a nutrient or nutraceutical, a recommendation to undergo a therapeutic treatment (e.g., antimicrobial treatment, anti-inflammatory treatment, etc.), a treatment to alter a therapeutic treatment (e.g., an ingested drug affected by the targeted microbe), a recommendation to increase physical activity, or the like, or combinations thereof. The survey can be generated and introduced to the individual subject responsive to sense signals generated by the physiological sensor, responsive to analysis of the biofluid 406 by the emissions analysis device 402, or combinations thereof.

The state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein can be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations can include software or other control structures. Electronic circuitry, for example, may have one or more paths of electrical current constructed and arranged to implement various functions as described herein. In some implementations, one or more media can be configured to bear a device-detectable implementation when such media hold or transmit device detectable instructions operable to perform as described herein. In some variants, for example, implementations can include an update or modification of existing software or firmware, or of gate arrays or programmable hardware, such as by performing a reception of or a transmission of one or more instructions in relation to one or more operations described herein. Alternatively or additionally, in some variants, an implementation can include special-purpose hardware, software, firmware components, and/or general-purpose components executing or otherwise invoking special-purpose components. Specifications or other implementations can be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications.

The foregoing detailed description has set forth various embodiments of the devices and/or processes via the use of block diagrams, flowcharts, and/or examples. Insofar as such block diagrams, flowcharts, and/or examples contain one or more functions and/or operations, each function and/or operation within such block diagrams, flowcharts, or examples can be implemented, individually and/or collectively, by a wide range of hardware, software, firmware, or virtually any combination thereof. In one embodiment, several portions of the subject matter described herein can be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution.

In a general sense, the various embodiments described herein can be implemented, individually and/or collectively, by various types of electro-mechanical systems having a wide range of electrical components such as hardware, software, firmware, and/or virtually any combination thereof and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Examples of electro-mechanical systems include but are not limited to a variety of consumer electronics systems, medical devices, as well as other systems such as motorized transport systems, factory automation systems, security systems, and/or communication/computing systems. Electro-mechanical as used herein is not necessarily limited to a system that has both electrical and mechanical actuation except as context may dictate otherwise.

In a general sense, the various aspects described herein can be implemented, individually and/or collectively, by a wide range of hardware, software, firmware, and/or any combination thereof and can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). The subject matter described herein can be implemented in an analog or digital fashion or some combination thereof.

With respect to the use of substantially any plural and/or singular terms herein, the plural can be translated to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “operably coupled to” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components can be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) can generally encompass active-state components and/or inactive-state components and/or standby-state components, unless context requires otherwise.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). If a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). Typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

This disclosure has been made with reference to various example embodiments. However, those skilled in the art will recognize that changes and modifications may be made to the embodiments without departing from the scope of the present disclosure. For example, various operational steps, as well as components for carrying out operational steps, may be implemented in alternate ways depending upon the particular application or in consideration of any number of cost functions associated with the operation of the system; e.g., one or more of the steps may be deleted, modified, or combined with other steps.

Additionally, as will be appreciated by one of ordinary skill in the art, principles of the present disclosure, including components, may be reflected in a computer program product on a computer-readable storage medium having computer-readable program code means embodied in the storage medium. Any tangible, non-transitory computer-readable storage medium may be utilized, including magnetic storage devices (hard disks, floppy disks, and the like), optical storage devices (CD-ROMs, DVDs, Blu-ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create a means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof are intended to cover a non-exclusive inclusion, such that a process, a method, an article, or an apparatus that comprises a list of elements does not include only those elements but may include other elements not expressly listed or inherent to such process, method, system, article, or apparatus.

In embodiments, the system 400 is integrated in such a manner that the system operates as a unique system configured specifically for function of the system 400 used to detect a measurable response elicited by interaction between at least one targeted microbe and the challenge agent 102 of the composition 100, and any associated computing devices of the system operate as specific use computers for purposes of the claimed system, and not general use computers. In embodiments, at least one associated computing device of the system operates as a specific use computer for purposes of the claimed system, and not a general use computer. In embodiments, at least one of the associated computing devices of the system is hardwired with a specific ROM to instruct the at least one computing device. In embodiments, one of skill in the art recognizes that the systems described herein (e.g., system 400) and associated systems/devices effect an improvement at least in the technological field of gut microbiota identification.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A composition for detecting at least one targeted microbe in a specific site of the gut, the composition comprising:
a challenge agent configured to elicit a measurable response by the at least one targeted microbe at the specific site in the gut, the measurable response detectable in a bodily gaseous emission; and
a controlled release system encompassing at least a portion of the challenge agent, the controlled release system configured to access the specific site of the gut.

2. The composition of claim 1, wherein the bodily gaseous emission includes at least one of respiratory gas, digestive gas, breath, or breath condensate.

3. The composition of claim 1, wherein the measurable response detectable in a bodily gaseous emission includes at least one of hydrogen, carbon dioxide, methane, nitric oxide, nitrogen, ammonia, a volatile organic compound, or a taggant.

4.-7. (canceled)

8. The composition of claim 1, wherein the challenge agent includes at least one saccharide.

9. (canceled)

10. The composition of claim 1, wherein the challenge agent includes at least one taggant, and the measurable response detectable in a bodily gaseous emission includes at least a portion of the taggant.

11. (canceled)

12. (canceled)

13. The composition of claim 1, wherein the challenge agent includes at least one substrate configured to interact with the at least one targeted microbe in a cell process.

14. The composition of claim 13, wherein the at least one substrate includes at least one of a metabolic substrate or an enzymatic substrate.

15. (canceled)

16. The composition of claim 1, wherein the specific site of the gut includes at least one of the mouth, throat, esophagus, stomach, duodenum, jejunum, proximal ileum, distal ileum, colon, or rectum.

17. The composition of claim 1, wherein the specific site of the gut includes a biomarker.

18.-24. (canceled)

25. The composition of claim 17, wherein the controlled release system includes at least one recognition element configured to recognize the biomarker.

26. (canceled)

27. The composition of claim 17, wherein the biomarker includes a biomarker of one or more resident microbes.

28.-30. (canceled)

31. The composition of claim 1, wherein the at least one targeted microbe includes one or more protozoa, one or more intracellular parasites or microparasites, one or more archaea, one or more fungi, or one or more bacteria.

32.-51. (canceled)

52. The composition of claim 1, wherein the controlled release system is configured for retention in a target gut site.

53.-164. (canceled)

165. The composition of claim 17, wherein the biomarker includes at least one of a gut biomarker, a cell surface marker, a pH marker, a chemical biomarker, a gastric fluid component, a digestive fluid component, an organ secretion, an extracellular matrix component, a cellular secretion, a mucus, or a mucus component.

166. The composition of claim 27, wherein the biomarker of one or more resident microbes includes at least one of a microbial surface molecule, a microbial functional biomarker, a microbial secreted molecule, or a microbial extracellular substance.

167. The composition of claim 1, wherein the controlled release system includes at least one of a capsule, an encapsulation, a controlled release coating, a controlled release matrix, a polymer, or a gelling agent.

168. The composition of claim 1, wherein the controlled release system includes at least one pH-sensitive component, the at least one pH-sensitive component including a first pH-sensitive component configured to access a first gut site and at least one additional pH-sensitive component configured to access at least one additional gut site.

169. The composition of claim 1, wherein the controlled release system is digestible by one or more resident microbes at the specific site in the gut and includes at least one of a synthetic component, a polysaccharide component, a lipid component, or a protein component.

170. The composition of claim 52, wherein the controlled release system includes at least one of a gastro-retentive component, an intestine-retentive component, a density that is less than a density of one or more digestive fluids in the target gut site, a bioadhesive component, a swellable component, an extendable component, an unfoldable component, a floating component, or an adherent component.

171. The composition of claim 1, wherein the controlled release system includes at least one of a recognition element configured to recognize at least one biomarker at the specific site in the gut, a substrate specific for a resident microbial enzyme, or an enzyme-degradable casing.
